(19) United States
(12) Patent Application Publication
(10) Pub. No.: US 2010/0004697 A1
(43) Pub. Date: Jan. 7, 2010

(54) Disengageable Anti-Return Device for a Rib Distractor

(76) Inventors: Frederic Fortin, Pessac (FR); Johann Robin, Begles (FR); Brice Sennequier, Pessac (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/083,184

(22) PCT Filed: Oct. 4, 2006
(86) PCT No.: PCT/FR2006/002225

§ 371 Date: Aug. 4, 2009

(30) Foreign Application Priority Data
Oct. 6, 2005 (FR) 0510207

(51) Int. Cl. A61B 17/58

(52) U.S. Cl. 606/86 R; 606/90

(57) ABSTRACT

The invention concerns a distraction device (4) for controlling the development of the deformation of a child's trunk during growth easily implantable into the human body and comprising rods (35, 36) coupled to the bones and mounted on central adjusting means (300) provided with a small hole (312) for engaging a small tool designed to adjust the distance separating the bone coupling means, said device (4) including a miniature casing (410) enabling the rods (35 and 36) to be translated in the direction of growth via a toothed wheel (311), said means being combined with an assembly of means: non-return catch (37), elastomer block (373), rigid cage (374) which enable the device (4) to be disengaged, tensioned and locked in one direction, while allowing growth to be monitored.

The invention concerns a refinement of the tensioning device which is placed between the ribs or vertebrae of a child whose growth has not ceased, and who presents deformations or malformations, particularly of the trunk and rachis.

At the present time, surgeons who specialize in this type of intervention use a distraction device. It allows congenital malformations to be corrected as well as those appearing in the course of growth, without prohibiting or blocking them. The present invention improves, by its new means, the implementation of the surgical treatment.

### PRIOR ART

This invention therefore presents new means which improve the patented invention published as No. FR 2794357 which allow for change in the deformation of a child's trunk to be managed in the course of growth and which is already characterized by its ease of implantation in the human body, and by its capacity for the correction in slight obstruction.

This invention of prior art includes: two rods mounted on a central means of adjustment provided with a small engagement hole for a small tool intended to adjust the distance that separates the means of attachment to the bones, said device being capable of being locked in a specified position by tightening several screws located on a central adjustment device. Although this invention does not necessitate surgery as invasive of that of even older prior art, such as the Campbell patent (WO/125553) which corrects by means of separation analogous to those of a notched belt including various spaces, but whose means do not look like those of the Fortin patent FR 2794357, nonetheless the fact remains that the miniaturization of the latter is fundamental to its implementation, the aim being to make a slight incision for its adjustment, once the device is implanted.

In the very old prior art of the Campbell patent, WO/125553, the opening made was large; in the case of the Fortin patent, it was highly reduced; it connects to the access to a central piece with a square shape, both at the time of emplacement and at the time of intervention for adjusting the device during growth.

In the scheme for adjusting and locking the device, the present invention goes even farther in non-invasive surgery at the time of adjusting the device which follows the growth. The present invention does permit the deformation to be corrected more rapidly, thanks to an opening even more reduced which allows the passage of a key a few millimeters in diameter in order to achieve adjustment of the central piece, which necessitates only a small intervention, very non-invasive and done under local anesthesia; it pushes the inventions of prior art into the technological background.

In the documents of prior art, in particular the anteriority of FR 2794357, the rods remain locked in both directions by the tightening of screws located at the top of the adjustment device; it is necessary to loosen them in order to translate the rods. In the present invention, there is a new means which is an anti-return device which does not prevent in any way the rods from being moved in the direction of growth, this movement being caused or not by the action of a mini-tool on the adjustment device.

Another document of prior art, Patent CA 2451977 (Campbell) describes a device including stepped holes in which a plate spring is positioned which provides locking of the device in a single direction. No disengagement is possible with this device, which is a great handicap during its emplacement or its maintenance, because this distraction device includes two sliding plates which are separate from one another and are locked by a cam located at the end of a plate spring by coming to lodge in a hole, thus preventing the plates from backing up. Nothing is provided to disengage such a device and make it operate in the reverse direction in order to facilitate its placement.

In all the following documents are of prior art:
US 2204/153067
U.S. Pat. No. 5,700,263
U.S. Pat. No. 3,900,025
DE 19500202
US 2004/097938.

A disengageable anti-return device is neither described nor suggested such as that precisely described in the present invention. For a professional person, if the anti-return function seems obvious, the function of disengaging is much less so and the means to achieve these two functions are not obvious either.

The combination of these documents which are of prior technological design cannot lead without some inventive thought to a disengageable anti-return device.

The figures aiding in understanding the invention are:

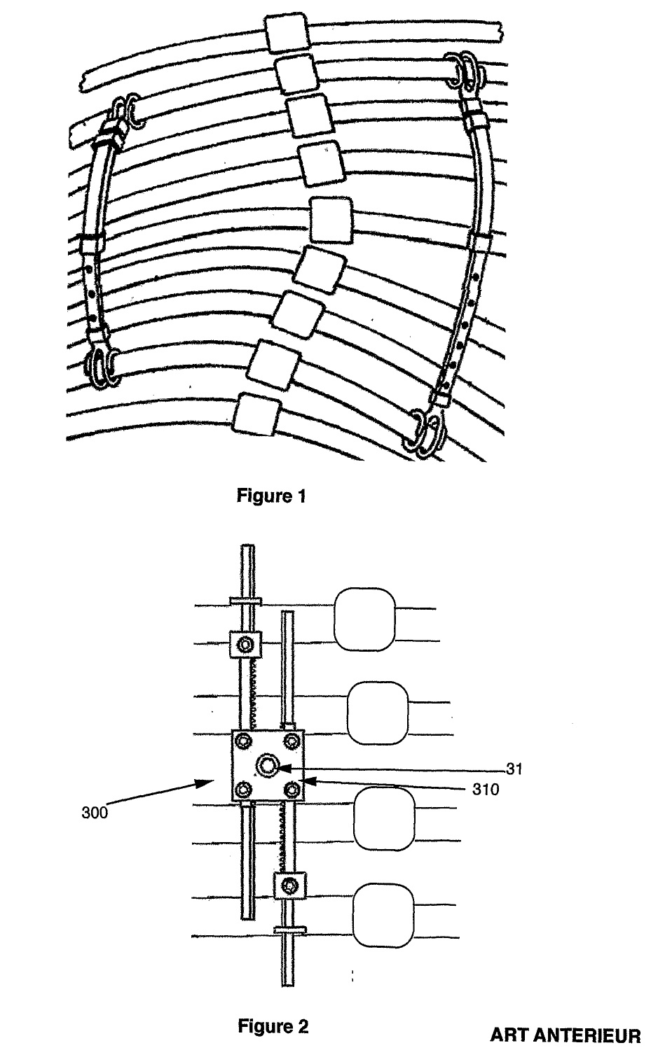

FIG. 1, Plate 1/5: Campbell device (very old prior art)

FIG. 2, Plate 1/5: first Fortin rib-distractor device (in recent prior art)

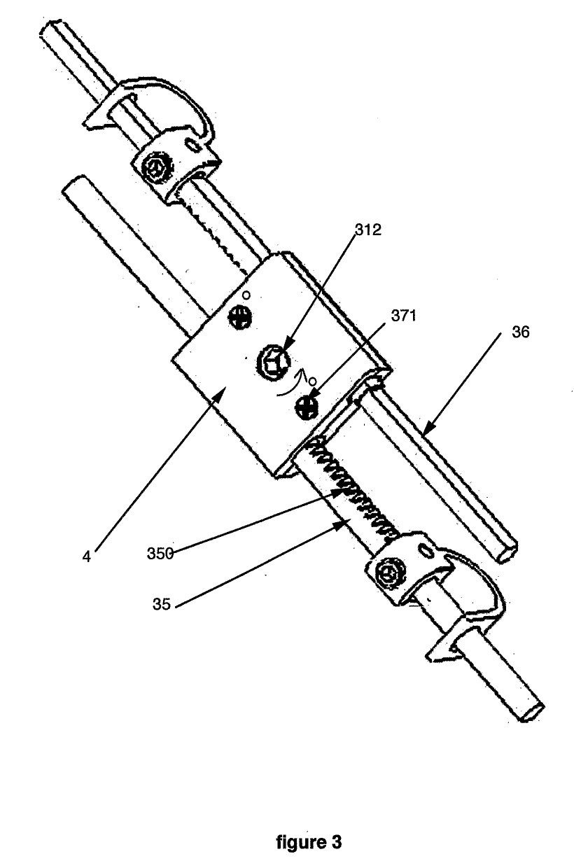

FIG. 3, Plate 2/5: isometric view of the new device

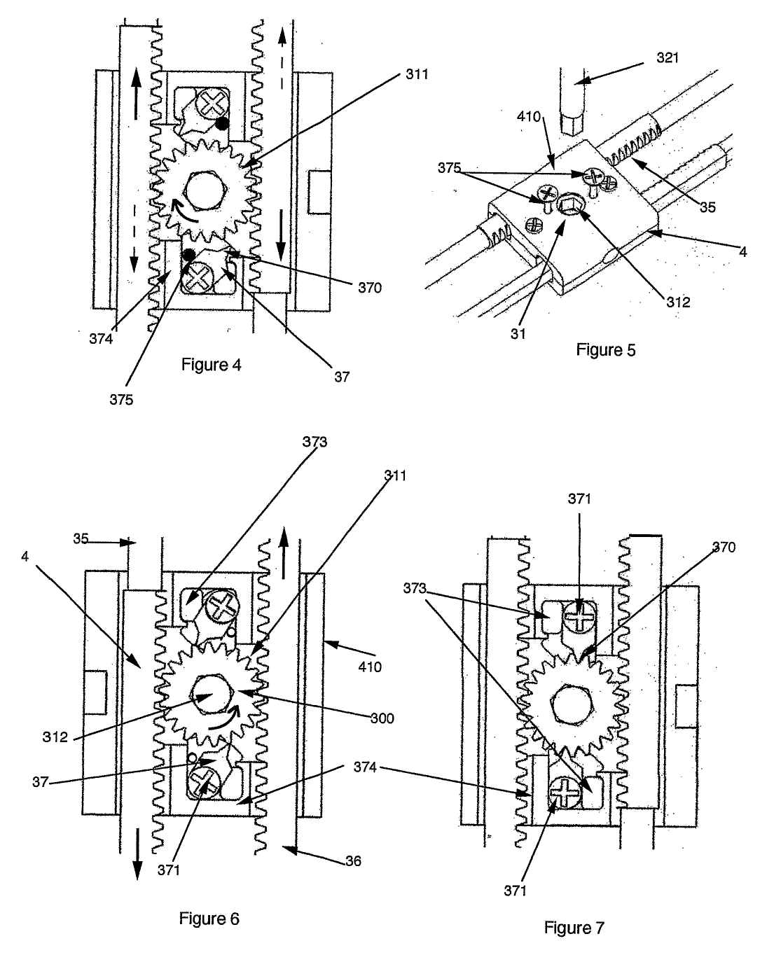

FIGS. 4, 5, 6, and 7, Plate 3/5 show more views of the new device, the subject of the invention:

FIG. 4: section of the central inner portion of the adjustment device with anti-return pawls and disengaged from the gear, which can turn in both directions

FIG. 5: view of the central internal portion of the new device, with the presence of an intervention tool of small diameter, at the time of implanting the device

FIG. 6: section of the central internal portion of the new adjustment device with pawls only allowing a single rotation direction

FIG. 7: section of the central internal portion of the new adjustment device with pawls locking the rotation of the gear in one direction and permitting its rotation in the other direction

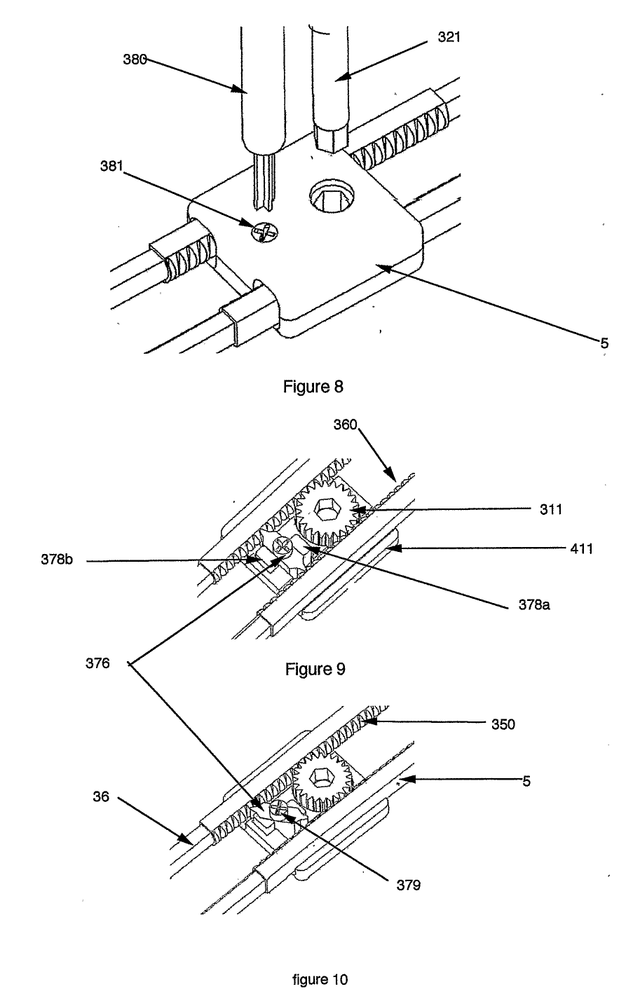

FIGS. 8, 9, and 10, Plate 4/5: perspective views of the device with a single pawl:

FIG. 8: device with mini-tools represented

FIGS. 9 and 10: views of the internal means on the device in a locked and unlocked position

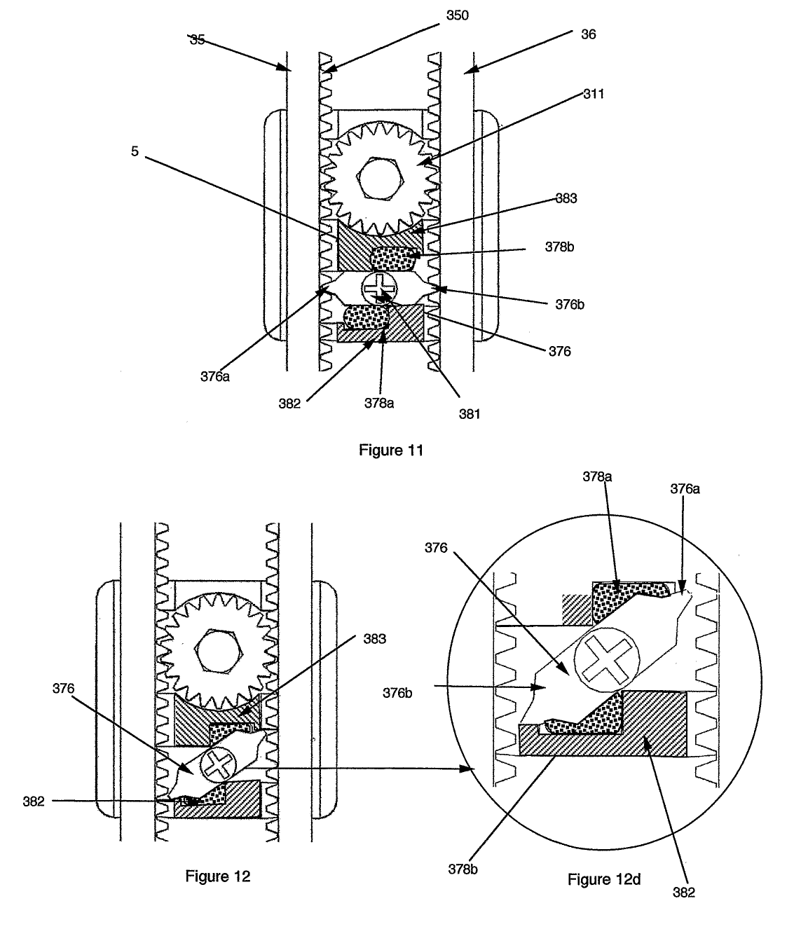

FIGS. 11, 12, and 12d of Plate 5/5: longitudinal sections: locked position (FIG. 11)
unlocked position (FIG. 12)

FIG. 12d: detail view of the pawl, unlocked position.

The present device 4 refining the distraction device of the first Fortin invention FR 2794357 is a disengageable anti-return device which includes known means and new means, subject of the present refinement. The known means corresponding to the Fortin invention of prior art comprise an adjustment device 300 having a central means 30 itself comprised of a mini-gearbox 310 at the interior of which a gear 311 turns centered on 310 and possessing a means 31 of initiating rotation.

This means may be a hole for the engagement of a key-type, male, six-sided tool 321, or any other equivalent means; its sole requirement is to be fitted into the gear 311.

In this mini-gearbox 310 at each side of the wheel 311 two rods 35 and 36 come to be positioned which can be placed in translation upon demand and in any direction thanks to the central means of adjustment 31 receiving the mini-tool 321.

The rods 35 and 36 possess a toothed part followed by preferably a smooth part, which allows the toothed parts to be engaged very easily and in a symmetrical manner, while subsequently permitting the meshing of the wheel 311. These rods are attached to the bones by means of attachment known to a professional person.

Before proceeding to the final adjustment, upon the systematic meshing of the rods, the mini-tool 321 can then be engaged in the adjustment device 31 in order to initiate distraction which is done in a quasi-automatic manner by turning the mini-tool 321 until resistance is felt to the advance of the rods. This operation is done of course after having positioned the device, the subject of the invention, on the bones. In this embodiment of prior art (FIG. 2), the adjustment and the locking of the device 31 take place by unlocking and locking several screws of the square gearbox 310. This requires, in order to perform this adjustment and this locking, an opening to be made matching the geometry of the miniaturized gearbox. The rods 35 and 36 are then locked in both directions.

In the present invention, the new device 4 comprises a new central mini-gearbox for adjustment 410 including the means known from prior art: a gear 311 centered on the gearbox 410 and possessing at one end of its axis a means 312 for putting the gear 311 into rotation which allows the advance of each rod 35 and 36 in an opposite direction, in distraction or in compression.

Once this new device 4 is implanted, the rods will only be moved in a single direction, that of growth, thanks to the presence of new means which are at least one pawl 37 including a tooth 370 always in contact with the gear 311 when the device is activated (FIGS. 6 and 7), said pawl 37 being found:
either at one side in contact with at least one elastomeric pad 373 which plays the role of a close-coil spring,
or on the other side supported by at least a rigid piece 374 integrated or brought into the gearbox 410, which is preferably a rigid rib, serving as a detent for the pawl and containing the elastomeric pad 373. Preferably two pawls 37 will be placed inverted (FIGS. 4, 5, 6, 7) located on either side of said gear 311 to support the mechanical loads transmitted to the device by the intermediary of the rods 35 and 36, said pawls 37 each including a tooth 370 able to pivot around an axis 371, these identical pawls 37 being able to take up three positions:

the first disengaged position (FIGS. 4 and 5): the pawls locked by a screw or a pin 375 are not in contact with the gear, the rods 35 and 36 can freely translate in both directions to facilitate the installation of the device 4 by the operator.

This position is obtained by setting into rotation in the direction that allows the pawl to be released from the gear 311, this maneuver is performed simultaneously on the one hand with the aid of the mini-tool 321 operating the gear 311 through the orifice 312 adapted to the end of the tool and on the other hand with another tool such as a screwdriver operating the rotation of the pawl 37 about its axis 371 accessible at the top of the gearbox 410. Finally the placement of a screw or mini-pins 375 between the piece 374 and the tooth 370 of the pawl permits by compressing the elastomeric pad 373 the prevention of the return of the pawl 37 toward the gear 311, the rods 35 and 36 are thus able to translate freely in both directions.

This operation may be performed beforehand, prior to implanting the device 4. The disengaged position is fundamental to the implantation of the distraction device. The operator does need to translate the rods back and forth in order to optimize its position and its fastening onto the bones.

Second Position: Putting the Anti-Return System into Operation

The device 4 having been attached to the bones, the means 375 are withdrawn completely, which has as a result the decompression of the viscoelastic pad 373 which plays the role of a spring-back force and pinning the tooth 370 against the gear 311 (FIG. 6).

With the aid of the mini-tool 321, the operator makes the gear 311 turn in order to place the device 4 in distraction in the only possible direction by causing the compression of the elastomeric pad 373. The gear 311 drives the rods 35 and 36 which translate in this direction, spreading apart the means of attachment to the bones.

Third Position: Self-Locking of the Device

As soon as the mini-tool 321 is withdrawn, the viscoelastic pads 373 (playing the role of a spring-back force) in decompressing push back the tooth 370 against the gear 311.

The rods 35 and 36 subjected to the tension exerted by the bones will drive the gear 311 in the direction opposite to that of growth, said gear 311 will in turn drive the tooth 370 of the pawl until said pawl 37 is located at the detent at the piece (or rib) 374, preventing the loss of distraction.

In the course of growth, the operator can easily put the device 4 back into distraction. It suffices to make a very small incision of a few millimeters in order to have access to the orifice 312.

He introduces the tool 321 and causes the gear 311 to turn in the only possible direction, thus putting the device back into distraction. Upon withdrawing the tool 321, the device 4 automatically prevents any return rearward of the rods 35 and 36.

These controls, and adjustments, of the tension of the device are facilitated by these new means described in the present invention.

In a second preferred embodiment, the new device 5 includes a single pawl or “double pawl” 376 (FIG. 9) which replaces the two pawls 37; this double pawl 376 does not operate on the gear 311.

This pawl 376 includes two opposing teeth 376 a and b placed symmetrically; this pawl pivots around an axis 379 which allows its disengagement and its engagement vis à vis the toothed parts 350 and 360 of the rods 35 and 36.

The disengageable anti-return device 5 likewise includes two viscoelastic pads 378 a and b playing the role of a spring-back force being exerted on the teeth 376 a and b by pinning them to the teeth of the grooved parts 350 and 360 of the rods 35 and 36.

The viscoelastic pads 378 a and b can be compressed by the rotation of the pawl around its axis 379 which permits the rods 35 and 36 to be capable of translation in both directions by rotating the gear 311 (FIG. 12).

The viscoelastic pads can be held in constant compression by means of a tool 380 introduced into the impression 381 of the axis 379 (FIG. 8), which permits the rods 35 and 36 to translate freely in both directions. The device 5 is then disengaged. This functionality is used for the emplacement of the distraction device.

As soon as the tool 380 is withdrawn and the viscoelastic pads are thus no longer held in compression, the teeth 376a and b of the pawl 376 are then pinned against the grooved parts 350 and 360 of the rods 35 and 36, which thus makes the device 5 engage.

The gear 311, contrary to preceding versions, has no connection with the double pawl 376, it has the sole function of driving the rods 35 and 36, while the pawl 376 allows the translation of the rods 35 and 36 in a single direction when it is engaged, and in both directions when it is disengaged. The viscoelastic pads 378a and b are lodged in the rigid compartments 382 and 383 located on the interior of the gearbox 411.

These compartments 382 and 383 also serve as a detent for the pawl 376.

The engagement is performed thus:

Under the effect of the spring-back force of the viscoelastic pads 378a and b, the teeth of the pawl 376a and b are each pinned against at least one of the teeth of the grooved parts 350 and 360 of the rods 35 and 36 which by moving in the direction of extension will compress the viscoelastic pads 378a and b, until at least one tooth 376a or b of the pawl 376 comes to be pinned to one of the next teeth or grooves 350 and 360 of the rods 35 and 36 which are thus able to advance bit by bit in a single direction.

When the rods 35 and 36 subjected to distraction are moved in the other direction, the teeth 376a and b drive the pawl 376 onto the detents 382 and 383 (FIG. 11). In this case, the device 5 automatically prevents the movement of the rods in this direction. In order to put the device back into distraction, after a period of growth, it suffices merely to introduce the first mini-tool 321 into the gear to cause the movement of the rods 35 and 36 which will translate in the only direction allowed by the pawl, thus permitting a minimal opening to be made in the back of the child and steadily apply a force sufficient to correct the deformation and successively stretch the distance between the bones.

In this embodiment, the gear 311 has no action upon the teeth 376a and b of the double pawl 376.

1-7. (canceled)

8. A distraction device for treating deformation of a spine of a growing patient, comprising:
two rods for attachment to bones, each rod including a plurality of teeth and an end portion having a bone-attaching portion; and
an adjustment system disposed between the two rods and engaging the rods such that the bone-attaching portions of the two rods are disposed on opposite sides of the adjustment system, the adjustment system including:
a disengagable anti-return device, which, when engaged, prevents movement of the bone-attaching portions relative to one another in a first direction, but allows movement of the bone-attaching portions relative to one another in a second direction.

9. The device of claim 8, wherein the anti-return device includes a gear configured to engage the teeth of the two rods.

10. The device of claim 9, wherein the anti-return device further includes at least one anti-return pawl configured to engage either the gear of the anti-return device or the teeth of at least one of the rods, thereby preventing relative movement of the bone-attaching portions of the rods in the first direction.

11. The device of claim 10, further including at least one tensioning member configured to force the at least one pawl into engagement with either the gear or the teeth of the rod.

12. The device of claim 11, wherein the at least one tensioning member includes an elastomeric material.

13. The device of claim 10, wherein the anti-return device includes two pawls located on opposite sides of the gear.

14. The device of claim 10, wherein each of the paws is attached within the anti-return device at a pivotable connection that allows each of the pawls to engage and disengage with either the gear or at least one of the two rods.

15. The device of claim 8, wherein, when the anti-return device is disengaged, the bone-attaching portions of the two rods are freely movable in the first direction and in the second direction.

16. The device of claim 10, further including a locking mechanism for locking the at least one pawl in a position disengaged from both the gear and the rods.

17. The device of claim 16, wherein the locking mechanism includes a pin or a screw.

18. The device of claim 10, wherein the at least one pawl includes a pawl having two toothed ends for engagement of the teeth of both rods.

19. The device of claim 18, further including two tensioning members configured to force the pawl into engagement with the teeth of the rods.

20. The device of claim 10, wherein the gear includes an opening configured to receive a tool for rotation of the gear.

21. The device of claim 10, wherein, when the anti-return device is engaged, the anti-return device prevents movement of the bone-attaching portions towards one another, but allows movement of the bone-attaching portions away from one another to increase the distance between the bone-attaching portions.

22. A distraction device for treating deformation of a spine of a growing patient, comprising:
two rods for attachment to bones, the rods being substantially parallel to one another, each rod including an end portion having a bone-attaching portion and a plurality of teeth disposed on an inward-facing surface of the rod;
a gear disposed between the two rods and including a plurality of teeth engaging at least one tooth of each of the rods; and
at least one pawl configured to engage either the teeth of the gear or the teeth of at least one of the rods to prevent movement of the bone-attaching portions relative to one another in a first direction, while allowing movement of the bone-attaching portions relative to one another in a second direction opposite the first direction.

23. The device of claim 22, further including at least one tensioning member configured to force the at least one pawl into engagement with either the gear or the teeth of the rod.

24. The device of claim 23, wherein the at least one tensioning member includes an elastomeric material.

25. The device of claim 22, wherein the anti-return device includes two pawls located on opposite sides of the gear.

26. The device of claim 22, wherein each of the pawls is attached within the anti-return device at a pivotable connection that allows each of the pawls to engage and disengage with either the gear or at least one of the two rods.

27. The device of claim 22, wherein, when the anti-return device is disengaged, the bone-attaching portions of the two rods are freely movable in the first direction and in the second direction.

28. The device of claim 27, further including a locking mechanism for locking the at least one pawl in a position disengaged from both the gear and the rods.

29. The device of claim 28, wherein the locking mechanism includes a pin or a screw.

30. The device of claim 22, wherein the at least one pawl includes a pawl having two toothed ends for engagement of the teeth of both rods.

31. The device of claim 30, further including two tensioning members configured to force the pawl into engagement with the teeth of the rods.

32. The device of claim 22, wherein, when the anti-return device is engaged, the anti-return device prevents movement of the bone-attaching portions towards one another, but allows movement of the bone-attaching portions away from one another to increase the distance between the bone-attaching portions.

33. The device of claim 22, wherein the gear includes an opening configured to receive a tool for rotation of the gear.

34. A method for treating a spine of a growing patient, comprising:
attaching an end portion of a first rod having a plurality of teeth to a first bone of the patient;
attaching an end portion of a second rod having a plurality of teeth to a second bone of the patient, wherein the first rod and the second rod are operatively connected to one another by a bone adjustment system positioned between the first rod and the second rod;
adjusting a distance between the end portion of the first rod and the end portion of the second rod; and
preventing movement of the end portion of the first rod and the end portion of the second rod relative to one another in a first direction while allowing movement of the end portion of the first rod and the end portion of the second rod relative to one another in a second direction opposite the first direction.

35. The method of claim 34, wherein adjusting the distance between the end portion of the first rod and the end portion of the second rod includes rotating a gear of the bone adjustment system, the gear having teeth that engage the teeth of the first rod and the second rod.

36. The method of claim 34, wherein rotating the gear includes inserting a tool into an opening of the bone adjustment system.

37. The method of claim 34, wherein preventing movement of the end portion of the first rod and the end portion of the second rod includes engaging an anti-return device of the bone adjustment system.

38. The method of claim 37, wherein engaging the anti-return device includes positioning at least one anti-return pawl for engagement with either the gear of the anti-return device or a toothed portion of at least one of the rods, thereby preventing relative movement of the end portion of the first rod and the end portion of the second rod in the first direction.

39. The method of claim 38, wherein engaging the anti-return device includes forcing the at least one pawl into engagement with either the gear or the toothed portion of at least one of the rods with a tensioning member of the anti-return device.

40. The method of claim 39, wherein enabling the at least one tensioning member to force the at least one pawl into engagement with either the gear or the teeth at least one of the rods includes removing a locking mechanism configured to lock the at least one pawl in a position disengaged from both the gear and at least one of the rods.

41. The method of claim 40, wherein the locking mechanism includes a screw or a pin.

42. The method of claim 37, wherein engaging the anti-return device prevents movement of the end portion of the first rod and the end portion second rod towards one another, but allows movement of the end portion of the first rod and the end portion of the second rod away from one another to increase the distance between the end portion of the first rod and the end portion of the second rod.

43. The method of claim 34, wherein the first bone is a first rib and the second bone is a second rib.

44. The method of claim 34, further including:
attaching an end portion of a third rod having a plurality of teeth to a third bone of the patient;
attaching an end portion of a fourth rod having a plurality of teeth to a fourth bone of the patient, wherein the third rod and the fourth rod are operatively connected to one another by a bone adjustment system positioned between the third rod and the fourth rod, and the third rod and the fourth rod are substantially parallel;
adjusting a distance between the end portion of the third rod and the end portion of the fourth rod; and
preventing movement of the end portion of the third rod and the end portion of the fourth rod relative to one another in a first direction while allowing movement of the end portion of the third rod and the end portion of the fourth rod relative to one another in a second direction opposite the first direction.

45. The method of claim 44, wherein the third bone is a rib and the fourth bone is a rib, and the third and fourth bones are on an opposite side of the spine from the first and second bones.

46. The method of claim 44, wherein preventing movement of the end portion of the third rod and the end portion of the fourth rod includes engaging an anti-return device of the bone adjustment system.
